(19) United States
(12) Patent Application Publication
(10) Pub. No.: US 2021/0087301 A1
(43) Pub. Date: Mar. 25, 2021

(54) METHOD FOR PRODUCING A THREE-DIMENSIONAL OBJECT BY A MULTIPHOTON PHOTOPOLYMERISATION PROCESS, AND ASSOCIATED DEVICE

(71) Applicants: ECOLE CENTRALE DE MARSEILLE, Marseille (FR); UNIVERSITÉ D'AIX-MARSEILLE, Marseille 7 (FR); Centre National de la Recherche Scientifique, Paris (FR)

(72) Inventors: Laurent Gallais-During, Pertuis (FR); Jean-Claude Andre, Nancy (FR)

(21) Appl. No.: 17/041,610

(22) PCT Filed: Mar. 27, 2019
(86) PCT No.: PCT/FR2019/050710

§ 371 Date: Sep. 25, 2020

(30) Foreign Application Priority Data
Mar. 28, 2018 (FR) 1852698

(51) Int. Cl. C08F 2/44; C08G 59/68; C08F 20/14; C08F 2/46; A61L 27/16; A61L 27/38; A61L 27/52; B33Y 10/00; B33Y 30/00; B33Y 70/10; B29C 64/135; B29C 64/268

(52) U.S. Cl. CPC C08F 2/44; C08G 59/68; C08F 20/14; C08F 2/46; A61L 27/16; A61L 27/38; B29K 2105/0002; B33Y 10/00; B33Y 30/00; B33Y 70/10; B29C 64/135; B29C 64/268; A61L 27/52

(57) ABSTRACT

A method for producing a three-dimensional object comprises the following operations: introducing a composition into a polymerization vessel; and polymerizing the composition by multiphoton polymerization, by means of a light source, in predetermined spots, in order to produce the three-dimensional object, the composition comprising at least one monomer, at least one filler and at least one photoinitiator, the composition having a transmittance per unit of length to the emission wavelengths of the light source, which is preferably higher than 75% and the at least one filler comprises nanoparticles.

### CROSS-REFERENCE TO RELATED APPLICATIONS

This application is a national phase entry under 35 U.S.C. § 371 of International Patent Application PCT/FR2019/050710, filed Mar. 27, 2019, designating the United States of America and published as International Patent Publication WO 2019/186070 A1 on Oct. 3, 2019, which claims the benefit under Article 8 of the Patent Cooperation Treaty to French Patent Application Serial No. 1852698, filed Mar. 28, 2018.

### TECHNICAL FIELD

The present disclosure relates to the field of three-dimensional printing. More particularly, the present disclosure relates to a method for producing a three-dimensional object and to a device for implementing this method.

### BACKGROUND

3D printing technologies have seen considerable interest since their first uses in the middle of the 1980s. The 3D printing techniques generally used are based on an additive-manufacturing principle, i.e., an object is obtained sequentially by superposing layers or by adding material sequentially or continuously.

A device for producing models of industrial parts is known from document FR 2567668. This device allows parts to be produced by scanning successive planes, horizontal planes for example, the scanning being carried out from the bottom of the tank containing a monomer liquid to its top.

The various 3D printing methods include more particularly fused deposition modelling (FDM), stereolithography (SLA), and selective laser sintering in which a laser agglomerates a powder layer. These various techniques have been considerably improved over these last few years to the point that they are no longer only used to produce prototypes, but increasingly also to produce functional objects.

One 3D printing technique, stereolithography by photo-polymerization of a liquid resin, was developed in 1984. This technique allows a three-dimensional object to be manufactured via a succession of layers of photo-polymerizable resin. The object is manufactured in a liquid resin bath. The resin is generally polymerized by radical polymerization, from a composition of acrylate monomers, or by cation polymerization, from a composition of epoxy monomers and of a photoinitiator that allows the polymerization under the effect of light radiation. Certain compositions applicable to single-photon stereolithography applications are commercially available. These compositions comprise monomers, typically acrylates or epoxies, and the photochemical initiator. In this technique, a process that is localized in space, with an amplification related to a chain reaction, is employed.

In this technique, a movable platform is generally submerged in a tank of liquid resin. This platform supports the object during its manufacture. The platform is positioned at a certain depth below the level of the resin. A laser beam is directed over the surface of the liquid resin to perform a suitable scan in order to photo-polymerize the resin and to thus form a slice of the three-dimensional object to be manufactured. After one slice has been processed, the platform drops a predefined distance corresponding to the thickness of a slice and the process is begun again for each slice of the object, thus allowing the complete three-dimensional structure of the object to be obtained. Once the stereolithography process has ended, the three-dimensional object is removed from the tank, washed and any holding elements are mechanically removed. Non-polymerized liquid resin present in the tank may subsequently be reused. Depending on the resin used, a last step, such as a baking step for example, in which the object is post-processed may be carried out in order to set it. In this technique, the deposition time of the various layers may be long if the technique is not stimulated, in particular in the case of use of viscous resins that exhibit a small volumetric shrinkage during polymerization. In the case of use of viscous resins with this technique, the use of doctor blades is often recommended in order to obtain a layer with a planar and uniform surface so as to ensure the good adhesion of the various layers to one another and also to prevent any collapse of the object during its manufacture.

These techniques have the drawback of not allowing layers that are very thin, and typically of the order of a few tens of μm in thickness, or even, under certain tricky conditions, smaller than one μm in thickness, to be produced. Specifically, such layers run the risk of moving or being torn off during the manufacture of the three-dimensional object. Moreover, these techniques for achieving 3D printing by superposition of layers do not allow objects that are complex or that require a high level of finishing to be manufactured. This is notably due to the viscosity of the resin and to its surface tension. As a general rule, a resin of low viscosity, generally of the order of a few tens of centipoise (cP), is preferred, because it corresponds to an optimum between the resolution of the object and the time taken to form and stabilize the layers.

To this is added the fact that it is also necessary to manufacture holder elements, such as one or more supports, rods for example, which will be removed once the three-dimensional object is taken out of the resin, to hold this object in the correct place during its construction in the tank. These holding elements prove to be necessary, on the one hand, because of the low viscosity of the resin and of its liquid nature, and on the other hand, because of the density of the polymerized material, which is generally slightly higher than that of the resin from which it is produced. An object created in a resin of low viscosity and without holding elements or supports will tend to move during the addition of a resin layer, this making the manufacture of the object difficult, or even impossible. Depending on the complexity of the object to be produced, certain of these supports may not be able to be easily removed and, in certain cases, the object will be unable to be manufactured using this 3D printing technique. The production of holding elements or manufacturing appendices, the sole aim of which is to allow the object to be manufactured, further increases the time taken to design, digitize, manufacture and finish the object.

Moreover, with 3D printing methods that use laser-induced fusion of a powder, the material making up the pulverulent medium has a consistency that gives it a structure close to that of a solid. Under these conditions, it is in principle not necessary to introduce supports into the method, even if layers are used. However, the high anisotropy associated with the fusing method means that certain manufacturers recommend using supports that compensate for mechanical tensions, in particular in the case of use of metal powders.

Furthermore, for objects requiring a surface of finished appearance, it is necessary to use a manufacturing resolution suitable for the desired surface finish or to carry out additional processing such as machining at the end of the printing method.

In order to overcome these limitations, it is possible to make use of multi-photon (SL2P) and notably two-photon photo-polymerization techniques. Two-photon photo-polymerization techniques have, for example, been developed by Shoji Maruo, Osamu Nakamura et Satoshi Katawa, “Three-dimensional microfabrication with two-photon-absorbed photopolymerization,” Opt. Lett. 22, pp. 132-134 (1997). These techniques consist in reaching directly, using a photon flux, which is advantageously formed by at least one focused laser beam, a designated location in a volume, a tank for example, in order to photo-polymerize the resin solely in this location. An object may thus be manufactured continuously by directing the focused laser beam into the volume of the tank containing the composition, without it being necessary to manufacture the object in slices or in successive layers. The production of three-dimensional objects by multi-photon photo-polymerization thus allows three-dimensional objects of high complexity to be produced with a high level of finishing, possibly for example of about a few tens of nanometers. These SL2P printing techniques require initiators that are capable of absorbing two photons sequentially or simultaneously to be used in order to form reactive species that allow the photo-polymerization to be initiated. Since the two-photon absorption requires, depending on the material, a high light density, of the order of about one hundred mJ/cm² at the focal point, the photo-polymerization is limited to the immediate vicinity of the focal point, where the light density is high enough to activate the initiator. One of the main advantages of two-photon stereolithography (SL2P) is that it allows three-dimensional objects to be manufactured without requiring the object to be manufactured in superposed layers or slices. In one- or two-photon photo-polymerization processes, for the polymerization of the resin to occur, it is necessary to cross a threshold related to local consumption of oxygen, which is a polymerization inhibitor. This allows spatial resolution to be improved relatively to the shape of the light beam. This effect is greater with two-photon absorption than with one-photon absorption.

This fixed resolution is associated with an elementary volume, called a voxel, produced by the laser pulse. Voxel is the acronym of “volumetric pixel.” If it is desired to achieve a good resolution without additional processing, i.e., when the object to be manufactured requires a high resolution, a very long manufacturing time is required and production costs that may be prohibitive are incurred. For this reason the SL2P technique is generally limited to objects of small size, often millimeter or even micron or nanometer size, and of simple shape with voxels that join up in the manufacturing process. In addition, this technique requires a high light density to be used at the focal point, which is generally of micron size and that is therefore not optimized for the manufacture of objects of centimeter size, or even of decimeter size, i.e., objects that may be inscribed in a volume typically comprised between 1 cm³ and 1000 cm³.

More recently, variable resolution SL2P techniques have been developed. A method for carrying out 3D printing by stereolithography is known from the document “Stereolithography with variable resolutions using optical filter with high contrast-gratings,” Li et al, J. Vac. Sci. Technol. B, Vol. 33, No. 6, November/December 2015. The variation in the resolution is obtained by using optical filters that modify the wavelength of the laser beam, thus allowing a pixel size variable between 37 μm and 417 μm to be obtained. One drawback of this method is that it uses two different wavelengths and therefore allows only two pixel sizes depending on the wavelength of the laser beam and on the optical filter. Moreover, this technique remains suitable solely for objects of micron size.

The document “Using variable beam spot scanning to improve the SL process,” Yi et al, Rapid Prototyping Journal, Vol. 19, No. 2, 2013, pp. 100-110, describes a variable-resolution stereolithography method. The variation in resolution is obtained via an optical device. This method allows three-dimensional objects of centimeter size to be formed. However, this method has many drawbacks and requires substantial optimization of the device depending on the objects to be produced. Although it is possible with this method to change the size of the voxel in two dimensions, it is not possible in the third dimension, perpendicular to the first two dimensions, depthwise for example.

More recently, methods referred to as bioprinting methods have been developed for the manufacture of living tissues, or even of organs. These methods are notably described in the following publications:
André J. C., Malaquin L., Guedon E. (2017), “Bio-printing; où va-t-on?,” Techniques de l'Ingénieur—ref RE2 68 V1, 23 pp. (2017);
Chua C. K., Yeong N. Y. (2015), “Bio-printing: principles and applications,” e-book World Scientific Ed.—Singapore;
Morimoto Y., Takeuchi S. (2013), “3D cell culture based on microfluidic technique to mimic living tissues,” Biomatter. Sci., 1, 257-264.

These bioprinting methods are additive manufacturing methods that employ living cells associated with carriers that are, for example, manufactured by stereolithography. One of the drawbacks of these methods is that they cause shearing movements during the placement of the successive layers. However, these movements are liable to damage the living cells and affect their survival.

### BRIEF SUMMARY

The objective of the present disclosure is to provide a method for producing a three-dimensional object by multi-photon, and notably two-photon, photo-polymerization, which method allows the aforementioned drawbacks of the prior art to be at least partially mitigated and allows objects that are nanometer size, or even centimeter or decimeter size, to be produced effectively.

Another objective of the present disclosure, which objective is different from the preceding objective, is to provide a method for producing an object of complex shape with fluid resins, and notably to provide a method allowing manufacturing of artefacts such as holding elements for example to be avoided.

Another objective of the present disclosure, which objective is different from the preceding objectives, is to provide a method for producing a three-dimensional object, in which method movement of the voxels during the production of this object may be averted and prevented.

Another objective of the present disclosure, which objective is different from the preceding objectives, is to provide a method for producing a three-dimensional object in which shear movements of the material from which the object is made and which could otherwise occur during the production of this object, are prevented.

In order to at least partially achieve at least one of the aforementioned objectives, one subject of the present disclosure is a method for producing a three-dimensional object, comprising the following operations:
introducing a composition into a polymerization tank,
polymerizing by multi-photon polymerization using a light source, in predefined locations, the composition, in order to produce the three-dimensional object, the composition comprising at least one monomer, at least one filler and at least one photoinitiator,
   the composition having a transmittance per unit length preferably higher than 75% at the emission wavelengths of the light source and the at least one filler comprising nanoparticles.

After the production of this three-dimensional object, the latter may be removed from the photo-polymerization tank then washed with a solution allowing non-polymerized composition to be removed from the three-dimensional object. This washing solution may, for example, be isopropanol or acetone.

By virtue of this method, the effectiveness with which three-dimensional objects of complex shapes that are currently inaccessible with fluid resins are produced is significantly increased. Specifically, produced voxels normally need to be supported. By virtue of the high viscosity of the composition, which is achieved by adding nanoparticles, manufacturing artefacts, such as for example supporting or holding appendices or elements that have to be removed once the three-dimensional object is finished in known prior-art methods, are avoided. By choosing high viscosities, the composition behaves substantially like a solid during the production of the three-dimensional object, this allowing potential movements of the voxels to be averted.

In addition, the use of nanoparticles allows the viscosity of the composition to be modified while avoiding problems related to the dispersion of light.

The producing method according to the present disclosure may furthermore comprise one or more of the following features, whether alone or in combination.

The composition has a viscosity higher than or equal to 0.30 Pa·s.

The nanoparticles have an average diameter smaller than or equal to 100 nm.

According to one aspect, the difference in the refractive indices of the nanoparticles and of the monomer is smaller than 0.4.

The composition may comprise from 10 vol % to 70 vol % of nanoparticles with respect to the volume of the composition.

According to one aspect, the fillers comprise a component that is soluble in the monomer.

The nanoparticles are made of a material chosen from: silica, glass, notably borosilicate glass or soda-lime glass, and an organic material that is insoluble in a constituent resin of the three-dimensional object.

According to one particular embodiment, the nanoparticles may be functionalized.

According to one aspect, the monomer is chosen from the following compounds: acrylic resins, L-lactic acid, glycolic acid, and caprolactones, these compounds possibly being used alone or in combination.

According to this aspect, the filler may furthermore comprise an additional constituent chosen from: living cells, a hydrogel chosen from collagen, fibrin, the alginate, chitin, chitosan, hyaluronic acid, poly-(2-hydroxyethyl methacrylate), polyvinyl alcohol and polyethylene glycol, whether alone or mixed.

According to one other aspect, the monomer is an acrylic monomer, in particular a multifunctional acrylic monomer.

According to this other aspect, the acrylic monomer is chosen from poly(ethylene glycol) diacrylates, tri(ethylene glycol) dimethacrylates, pentaerythritol tetraacrylates, 1,6-hexanediol diacrylate, or a combination of these compounds.

Again according to this other aspect, the one or more photoinitiators are chosen from: aromatic ketones, aromatic derivatives, eosin Y, or other xanthene dyes.

According to one variant, the composition may comprise at least one epoxy monomer.

According to this variant, the photoinitiator is an onium salt.

According to one particular embodiment, the multi-photon polymerization is carried out using a laser beam and the polymerization spatial resolution is set by placing an optical diffuser, notably of between 1° and 20°, in the laser beam, the optical diffuser being configured to modify the depth of the field of the laser beam.

According to one other particular embodiment, the three-dimensional object comprises an external surface and an internal volume and localized locations in the internal volume are polymerized with a lower resolution than locations forming the external surface of the three-dimensional object.

According to this other particular embodiment, various segments of the three-dimensional object are successively polymerized in various tanks each containing a specific composition allow a predefined voxel size, or even predefined functionalities, to be obtained.

According to this other embodiment, the internal volume is polymerized in a first tank containing a first composition comprising first fillers taking the form of nanoparticles allowing a first voxel size to be obtained and the external portion of the three-dimensional object is polymerized in a second tank containing a second composition comprising second fillers taking the form of nanoparticles or no filler allowing a second voxel size smaller than the first voxel size to be obtained.

Another subject of the present disclosure is a device for producing a three-dimensional object by multi-photon and notably two-photon photo-polymerization, comprising:
a light source that emits a laser beam,
a polymerization tank containing a composition comprising:
   at least one monomer,
   at least one filler comprising nanoparticles such as defined above, and
   at least one photoinitiator,
      the composition having a transmittance per unit length preferably higher than 75% at the emission wavelengths of the light source,
a device for focusing the laser beam and for setting its numerical aperture,
a moving unit in order to allow the focal region of the laser beam to be moved inside the tank to preset locations in order to produce the three-dimensional object, and
a polymerization-resolution setter comprising at least one optical diffuser moveably mounted on a holder so as to be placeable on the optical path or outside of the laser beam, in order to set the polymerization resolution.

### BRIEF DESCRIPTION OF THE DRAWINGS

Other features and advantages of the present disclosure will become more clearly apparent on reading the following non-limiting description, which is given by way of illustration, and the appended drawings, in which:

FIG. 1 is a simplified schematic of a setup of a device for producing a three-dimensional object,

FIG. 2 is a detailed schematic of a composition used to produce a three-dimensional object,

FIG. 3 illustrates, in a table, a non-exhaustive list of monomers able to be used in the composition,

FIG. 4 illustrates in a table a non-exhaustive list of photoinitiators able to be used in the composition,

FIG. 5 illustrates a conventional ion-polymerization mechanism with steps of initiation, propagation and transfer,

FIG. 6 is a schematic of multifunctional monomers of cross-linked systems, which are insoluble in an initial resin,

FIGS. 7A and 7B are photographs showing voxels obtained, on the one hand, in the case of a Gaussian beam, and on the other hand, by placing a diffuser before the entrance of an objective in order to control the depth of field of the Gaussian beam, the two FIGS. 7A and 7B being to the same scale, respectively,

FIG. 8 is a schematic allowing the method for producing a three-dimensional object according to one particular embodiment to be illustrated,

FIG. 9 is a graph showing the measurement of the diameter of the beam as a function of the distance with respect to the objective in the case of the objective alone and in the case of a 1° and 10° diffuser,

FIG. 10 is a schematic representation of a flowchart illustrating a method for producing a three-dimensional object, and

FIG. 11 is a schematic representation in perspective of an object manufactured using the method of FIG. 10.

FIG. 12 is a photograph showing a dodecahedron obtained using the method of the present disclosure.

FIG. 13 is a photograph showing a bicycle obtained using the method of the present disclosure.

FIG. 14 is a photograph showing an object having the double-helix shape of a segment of DNA, this object being obtained using the method of the present disclosure.

### DETAILED DESCRIPTION

In the various figures, identical elements have been designated by the same reference numbers.

The following embodiments are examples. Although the description refers to one or more embodiments, this does not necessarily mean that each reference regards the same embodiment or that the features apply only to one signal embodiment. Single features of various embodiments may also be combined and/or interchanged to produce other embodiments.

In the following description, reference is made to first and second photo-polymerization tanks, to first and second compositions, to first and second fillers, and to first and second voxel sizes. It is merely a question of an index allowing elements that are similar or of same nature or structure but that are not identical to be named and differentiated. This indexation does not imply that one element has priority over another and such denominations could easily be interchanged without departing from the context of the present description. This indexation also does not imply an order in time, for example, with respect to the operation of the device for producing or even to the method for producing the three-dimensional object.

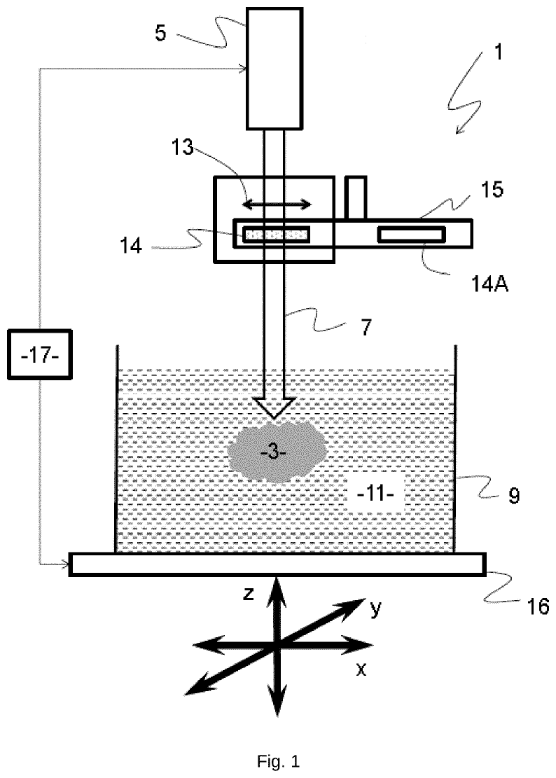

With reference to FIG. 1, a producing device 1 for producing a three-dimensional object 3 by multi-photon and notably two-photon photo-polymerization is shown.

This producing device 1 comprises a light source 5 that emits a laser beam 7 and a polymerization tank 9, which forms a polymerization reactor, and which contains a composition 11.

The light source 5 may, for example, be a laser, in particular a pulsed laser, and notably a femto/picosecond laser that, for example, emits at a wavelength of 1030 nm and that is coupled, where appropriate, to non-linear optical crystals allowing, via a non-linear effect, the frequency of the laser beam 7 to be doubled or tripled, in order to obtain a wavelength of 515 nm and/or 343 nm. The light source 5 therefore emits, according to this example, a pulsed light beam. The choice of the light source 5 may depend on the absorption of the composition 11, which may contain colored additives for example. Thus, other types of notably pulsed light sources 5 may be used.

The choice of the multi-photon and notably two-photon photo-polymerization wavelength is dependent on the choice of the photoinitiator and on its capacity to initiate the reactive species under the effect of the laser radiation.

Typically, the output diameter of the laser beam 7 may be about 2.5 mm, its divergence 0.6 mrad and its polarization linear.

The energy per pulse typically has a duration of 500 fs and is comprised between 40 μJ and 2 mJ, and the repetition frequency of the pulses may reach 300 kHz, but may rather be located around 1 kHz. Another light source 5 may be used provided that the wavelength of its laser beam 7 is suitable and that the instantaneous power of the laser allows the multi-photon and notably two-photon photo-polymerization of the composition 11 that is found in the polymerization tank 9 to occur.

The producing device 1 furthermore comprises a device 13 for focusing the laser beam 7 and setting its numerical aperture, this device being placed on the optical path of the laser beam 7. This focusing device 13 may formed by one or more optical components, notably an objective for focusing the laser beam 7 inside the composition 11 and setting the numerical aperture of the laser beam 7.

Optionally, the producing device 1 may comprise a polymerization-resolution setter comprising at least one optical diffuser 14 placed on the optical path of the laser beam 7 in order to allow the depth of field of the laser beam 7 to be controlled. To this end, the producing device 1 comprises a rotary holder 15 with a through-hole 14A for focusing the laser beam 7 without modification of the beam in the composition 11 and housings in which are respectively mounted various diffusers 14 allowing the depth of field to be set. Thus, it is possible, as has already been eluded to, to make the size of the voxels vary. The holder 15 with its one or more diffusers 14 and the through-hole 14A allows the dimensions of the voxels to be adjusted and a variable resolution to be obtained in the manufacturing process by adjusting the focusing optic and the instantaneous power of the laser beam 7.

The polymerization tank 9 is, for example, placed on a moving unit 16 that is moveable along the axes x, y and z (shown in FIG. 1) in order to allow the focal region of the laser beam 7 to be moved inside the tank 9 and therefore the composition 11 to be polymerized in preset locations in order to produce the three-dimensional object 3. It will therefore be understood that, according to this particular embodiment, it is the polymerization tank 9 that is moved in order to allow the focal point of the laser 5 to be positioned in the locations to be photo-polymerized and not the focal point of the laser 5. To achieve this, the moving unit 16 is motorized in order to allow it to move. This moving unit 16 is connected, just like the laser 5, to a control unit 17, which controls both the operation of the laser 5 and the positioning of the moving unit 16.

According to one variant (not shown here) moveable mirrors are placed on the optical path of the laser beam 7 in order to direct the laser beam 7 to the locations that must be photo-polymerized and a system for focusing the laser and for setting its numerical aperture, allowing the focal point to be moved over the propagation axis, is provided. In this case, the moveable mirrors are connected to a control unit in order to allow the laser beam 7 to be directed.

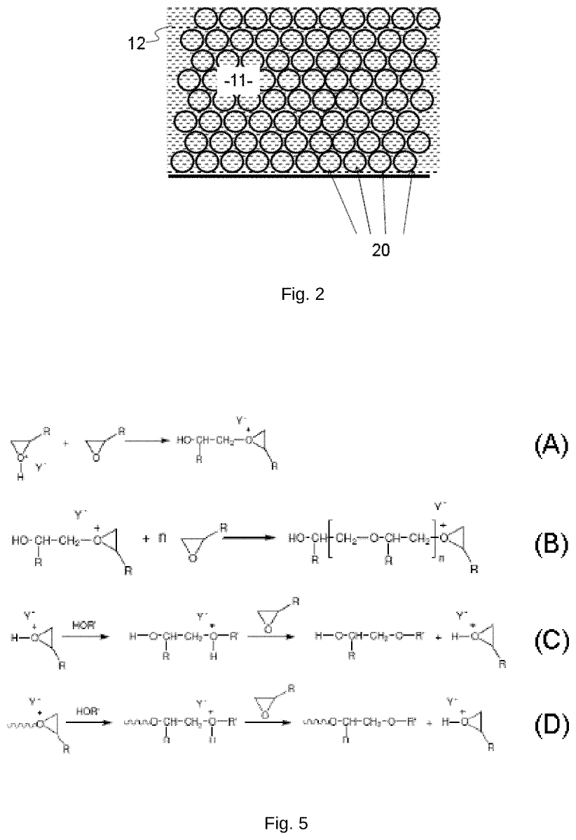

With reference to FIG. 2, the composition 11 for producing the three-dimensional object 3 via a multi-photon photo-polymerization process is shown in a simplified and schematic way. The composition 11 comprises at least one monomer 12, at least one filler 20 comprising nanoparticles, and at least one photoinitiator.

The monomers 12 are transparent at the preset wavelength of the pulsed source that serves for the photo-polymerization. These monomers 12 have a refractive index nₘₒₙₒₘₑᵣ at the preset photo-polymerization wavelength of the laser 5. By a transparent medium or material, what is meant is that the laser beam 7 may pass, at least partially (i.e., it may be weakly absorbent) through this medium in contrast to an opaque medium or material.

By filler 20, what is meant is a material or substance in the broad sense that is added to the composition 11, but that does not participate in the polymerization reaction. The filler 20 may be considered inert with respect to the polymerization. The fillers 20 are nanoparticles that are transparent or very weakly absorbent at the preset wavelength of the pulsed source that serves for the photo-polymerization. These fillers 20 have a refractive index milers at the preset photo-polymerization wavelength.

Since the irradiation occurs at the preset wavelength λ, this wavelength may be partially absorbed during its trip through the tank containing the polymerizable resin. If a corresponds to an absorption coefficient (measured in m⁻¹), the relative energy at a distance d (d≠0) from the point where the beam entered is given by the formula exp (−α·d)<1. The loss of photons may be prejudicial to the method since it is the square of the instantaneous power that sets the polymerization rate. However, it is possible to increase the power of the laser in order to compensate for the effects of the absorption of incident photons. The transmittance limit is, for example, 75%, this corresponding to a loss of polymerization efficiency of 0.56, which is acceptable although the time taken to manufacture a part using the method is increased accordingly.

Thus, the composition 11 has a transmittance per unit length higher than 75% at the emission wavelengths of the laser 5. According to the particular embodiment shown here, the unit of length corresponds to a dimension of the polymerization tank 9, and more precisely to a height of the polymerization tank 9, which is the dimension along the z-axis (as shown in FIG. 1). However, according to other variants (not shown here) the unit of length may, for example, be a metric unit, such as, for example, 1 decimeter or 1 meter.

When a light beam illuminates a dispersion, namely here the composition 11, characterized by its refractive index, the light undergoes a scattering/absorption process that is dependent on the wavelength of the incident light and on the optical properties of the continuous and dispersed phases. The scattering/absorption effects thus induce an attenuation of the incident light in the initial direction of the incident beam. The intensity of the scattered light depends on the scattering direction with respect to the direction of the incident beam, on the polarization of the incident light and on the characteristics of the scattering medium.

For a low number of scatterers, most of the incident light passes through the medium without undergoing scattering. For a high attenuation of the incident beam (multiple scattering regime), the light scattered by a particle, which corresponds to any type of insolubilized particle present in the medium, is a secondary source for neighboring particles. In the case of a particle, the waves scattered by the various regions of the material interfere with one another. The interaction of an incident wave with a spherical, uniform, isotropic and non-magnetic particle of diameter d in a non-absorbent medium is described by Maxwell's equations.

Only solution of Maxwell's equations then allows the intensity scattered by the particle in any direction to be determined. Mie was the first to solve the problem for uniform dielectric spheres and to obtain an analytical solution for a spherical particle of arbitrary size. The theory of Mie provides a rigorous solution to Maxwell's equations. At small scattering angles, the intensity of the scattered light is particularly high in the case of large particles. In addition, the angular variation in the light scattered varies monotonically for non-absorbent particles and decreases with the scattering angle because of destructive interference in the backward direction. Light-absorption effects tend to suppress scattering lobes and the fine structure of the radiation pattern. The angular dependence of the scattered light is less pronounced for small particles, and thus information may be obtained on the size of the particles.

The intensity of the scattered light also depends on the values of the refractive indices of the particles and of the surrounding medium, and on the wavelength of the incident light. Depending on the size of the particles with respect to the wavelength of the incident light, approximations however allow effects due to scattering of the light to be satisfactorily taken into account.

The theory of Rayleigh describes the scattering of light by particles the dimensions of which are very small with respect to the wavelength of the incident light (particle diameter smaller than one tenth of the wavelength of the incident beam). In this case, the incident electric field with which a particle is illuminated may be considered to be uniform in the scatterer and the intensity of the scattered light is then proportional to the square of the volume of the particle. For non-absorbent particles, the intensity scattered per unit volume may be expressed in the following way:

$$I = {I}_{0} \frac{1 + {cos}^{2} θ}{2 {r}^{2}} {( \frac{2 Π}{Λ} )}^{4} ( \frac{{m}^{2} - 1}{{m}^{2} + 2} ) {( \frac{d}{2} )}^{6} N$$

where I₀ is the intensity of the incident light of wavelength λ, θ is the scattering angle, m=n_{particle}/n_{medium} is the index ratio (choice of index) between the particle and the host medium, here the monomer 12, and N is the number of non-absorbent particles per unit volume. When the size of the particles is very large with respect to the wavelength of the incident light (particle diameter larger than 10-20λ the scattered intensity is essentially concentrated in the forward direction. The approximations of geometric optics are then used to describe the scattering of the light.

The preceding expression allows the absorption cross section (σ) of a set of particles to be obtained:

$$σ = \frac{2 {π}^{5}}{3} \frac{{d}^{6}}{{λ}^{4}} ( \frac{{m}^{2} - 1}{{m}^{2} + 2} ) N$$

Considering a configuration m=1.05, d=10 nm, λ=1030, and 1 particle per 10 nm³, a σ of about 10⁻³ is obtained, allowing scattering effects to be neglected.

The composition 11 is therefore transparent, at least to a first approximation, at the wavelength of the light source 5. It is therefore not necessary to modify the pair consisting of the filler 20/monomer 12 to achieve a low index difference. The difference between the refractive index nₘₒₙₒₘₑᵣₛ of the monomer 12 and the refractive index n_{fillers} of the fillers 20 is smaller than 0.4, and preferably smaller than 0.05 (|nₘₒₙₒₘₑᵣₛ−n_{fillers}|<0.05), and more particularly smaller than 0.01 (|nₘₒₙₒₘₑᵣₛ−n_{fillers}<0.01), or indeed the refractive index of the monomers 12 and the refractive index of the fillers 20 may be equal (|nₘₒₙₒₘₑᵣₛ−n_{fillers}|=0). Choosing a refractive-index difference that is low or even zero allows any effects due to scattering of the laser beam 7 in the composition 11, and notably at the interfaces between the monomers 12 and the fillers 20 at the wavelength emitted by the light source 5, to be suppressed, in so far as the size of the fillers 20 affects the movement of the light in the case of fillers 20 of size very much larger than the wavelength.

The refractive index n_{composition} of the composition 11 is the result of all its components Cᵢ (monomers 12 and fillers 20) in their proportions in the composition 11. Thus, if V_{R} is the density of the composition 11 and V_{Ri} the density of each of the components Cᵢ, and αᵢ is a rational number comprised between 0 and 1, the following are obtained:

$${V}_{R} = {\sum }_{i = 1}^{m} {α}_{i} {V}_{Ri}$$

$${\sum }_{i = 1}^{m} {α}_{i} = 1$$

$${n}_{composition} = {\sum }_{i = 1}^{m} {α}_{i} {n}_{1 i}$$

nᵢⱼ being the refractive index of the component Ci, and
i, j and m being whole numbers, m corresponding to the number of constituent components Cᵢ of the composition 11.

In this case, it will be understood that by adjusting the proportions of the components Cᵢ it is also possible to adjust the refractive index n_{composition} of the composition 11 (and also to adjust, on the one hand, the refractive index of the one or more monomers 12, and on the other hand, the refractive index of the filler 20) provided that at least one refractive index nᵢⱼ of the component Cⱼ (i≠j) is, for example, higher than the second refractive index n₂.

The viscosity of the composition 11 may be adjusted, via the choice of the volume percentage of filler 20, and in particular of nanoparticles, to a value higher than 0.30 Pa·s and preferably comprised between 0.30 and 5.00 Pa·s (pascal·second), in order to obtain a stable or petrified composition, i.e., in which the object in the process of being manufactured, but also the filler 20, does not move. The viscosity of the composition 11 is adjusted, notably depending on the manufacturing time of the three-dimensional object 3 or even depending on the size, and in particular on the radius or equivalent radius when the three-dimensional object 3 is not spherical, of the three-dimensional object 3 to be manufactured, so that the movement of this three-dimensional object 3 during its manufacture by photo-polymerization is negligible. Furthermore, heating the composition 11 slightly allows its viscosity to be decreased and the tank to be filled while avoiding the potential presence of air bubbles. This principle may be applied to the separation of the three-dimensional object 3 once it has been manufactured from the composition 11. By making use of the high viscosity of the composition 11, it is possible to avoid manufacturing artefacts such as, for example, supporting or holding elements. Specifically, in hydrodynamics, the Reynolds number Re expresses the relative importance of viscosity effects and of inertia effects. It is given by the following relationship:

Re=ρVD/μ

where ρ is the density of the fluid, μ is the viscosity, and V and D are a characteristic speed and length of the flow in question. Flows at low Reynolds numbers are characterized by the predominance of effects due to viscosity over those due to inertia. Thus, by choosing high viscosities, the composition practically behaves as a solid, this preventing movement of the voxels.

The filler 20 takes the form of nanoparticles, which are, for example, formed of nanoparticles that are insoluble or that comprise a component that is soluble in the monomer 12, such as, for example, soluble macromolecules such as, for example, linear acrylic polymers solubilized in an acrylic resin, in the composition 11. The average size of the nanoparticles is very much smaller than the excitation wavelength of the laser beam 7.

The ability of a substance, called a solute, to solubilize in another substance, called a solvent, in order to form a uniform mixture, called a solution, is the “solubility.”

According to one particular embodiment, when the filler 20 comprises insoluble nanoparticles, the volume percentage of filler 20 in the composition 11 is comprised between 10 vol % and 70 vol % with respect to the volume of the composition 11, notably between 30 vol % and 60 vol % and more particularly between 40 vol % and 50 vol %. More precisely, 100 vol % represents the total volume of the composition 11 and this volume is separated into various volumetric proportions for each of the constituents of this composition 11.

Furthermore, the nanoparticles have an average diameter smaller than or equal to 100 nm, notably comprised between 7 nm and 70 nm and more specifically of 10 nm. The maximum size of the nanoparticles is chosen depending on the diffraction limit of the incident wavelength of the laser beam 7, i.e., about one tenth of the incident wavelength output by the light source 5.

The nanoparticles are, for example, made of a material chosen from: silica, such as, for example, fused silica, glass, notably borosilicate glass or soda-lime glass, an organic material that is insoluble in a resin from which the three-dimensional object 3 is made, such as, for example, acrylic or epoxy nanoparticles. Alternatively or in addition, the nanoparticles may be functionalized in order to modify their chemical affinity with the monomers 12 or even to endow them with particular properties.

According to one particular embodiment, the nanoparticles are monodisperse, i.e., they all have the same diameter. Alternatively, these nanoparticles may be of variable sizes, but they meet the diffraction constraint defined above.

The use of monodisperse nanoparticles allows in certain configurations the size of the voxels to be defined. Specifically, when the diameter of the nanoparticles is larger than the focal volume of the laser beam 7, the size of the voxel is no longer defined by the focal volume of the laser beam 7 but by the diameter of the nanoparticles. In particular, the shape of the voxels may thus be made perfectly spherical, even though the focal volume of the laser beam 7 is not, the laser beams 7 serving solely to agglomerate, by virtue of the photopolymer created, at the focal point the nanoparticles, which then define the size of the voxels.

According to another embodiment, the composition 11 is a petrified composition, for example a composition comprising, by way of monomers, oligomers of high molecular weight allowing a composition that is solid or quasi-solid at room temperature to be obtained, so that it is thus possible to photo-polymerize an object without having to produce holding or supporting appendices. Before and/or after photo-conversion, the composition 11 may be heated above the melting point of the resin in order to allow the resin to the introduced into the photo-polymerization tank 9 in a liquid (or viscous) form and/or in order to separate the object from the composition 11 from which it is obtained. This has the advantage of significantly decreasing the time taken to produce the three-dimensional object 3 and of allowing highly complex parts that would be difficult, or even impossible, to manufacture with other methods requiring supporting appendices to be produced to be created.

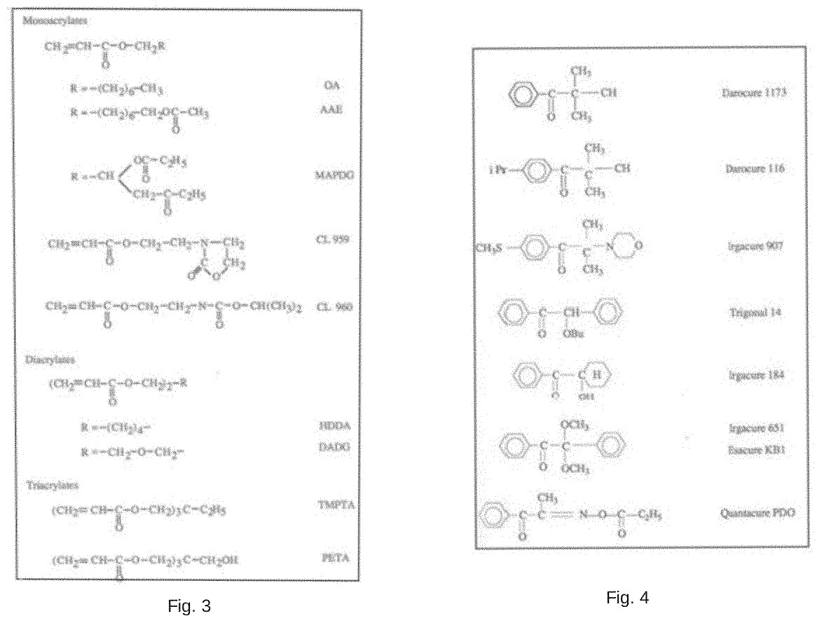

In the case of a liquid composition, the monomers 12 present in the composition 11 are monomers 12 commonly used in 3D printing by mono- or multi-photon photo-polymerization. These monomers 12 are, for example, acrylic monomers, and more specifically acrylates. Furthermore, these acrylic monomers may be multifunctional. A non-exhaustive list of monomers 12 that may be used in the composition 11 is provided, by way of reference, in FIG. 3.

It will be noted that as a result of the viscosity of the composition 11 (higher than or equal to 0.05 Pa·s=0.5 poiseuilles=50 cP) the filler 20, which, in particular, takes the form of nanoparticles, is almost petrified in the composition 11, i.e., the movement of the nanoparticles is small or almost zero in a time corresponding to the length of time taken to produce a three-dimensional object 3. Under these conditions, the effects of the force of gravity on the nanoparticles are negligible. No noteworthy sedimentation has been observed.

Preferably, the acrylic monomer is chosen from poly(ethylene glycol) diacrylates, tri(ethylene glycol) dimethacrylates, pentaerythritol tetraacrylates, 1,6-hexanediol diacrylate, or a combination of these compounds.

The radical photoinitiators contained in the composition 11 must allow the polymerization to be initiated at the preset photo-polymerization wavelength. There are, depending on the operating conditions, a high number of suitable photoinitiators that a person skilled in the art will be easily able to select from. The photoinitiators below are given by way of non-limiting example. It is typically a question of aromatic ketones, such as, for example, 2,2-dimethoxy-1,2-phenylacetophenone (DMPA), which is sold under the name IRGACURE 651®, aromatic derivatives, eosin Y for photo-polymerizations in the visible domain, or thermal initiators such as benzoyl peroxide for photo-polymerizations in the infrared domain, or even of other xanthene dyes. Photoinitiators particularly suitable for the method according to the present disclosure are shown in FIG. 4 and sold under the trade names DAROCURE 1173® and 116®, QUANTACURE PDO®, IRGACURE 184®, 651®, and 907®, and TRIGONAL 14®.

Preferably, the radical photochemical initiator is the DMPA sold under the name IRGACURE 651®.

According to another embodiment, the method of the present disclosure uses an ionic, and, for example, cationic, photo-polymerization mechanism. In which case, the monomers 12 present in the composition 11 are, for example, epoxy monomers and the photoinitiator is an onium salt, such as, for example, HODORSIL 2074®. The following reference: Vairon J-P & al, “Industrial Cationic Polymerization: An Overview in Cationic Polymerizations,” Matyjaszewszki, K., Ed., Marcel Dekker: New York, N.Y., USA, 1996, pp. 683-750 gives a list of various photo-chemical initiators usable in the method forming the subject matter of the present disclosure.

FIG. 5 illustrates a conventional ionic polymerization mechanism with the following steps: initiation (A), propagation (B) and (C), transfer (D).

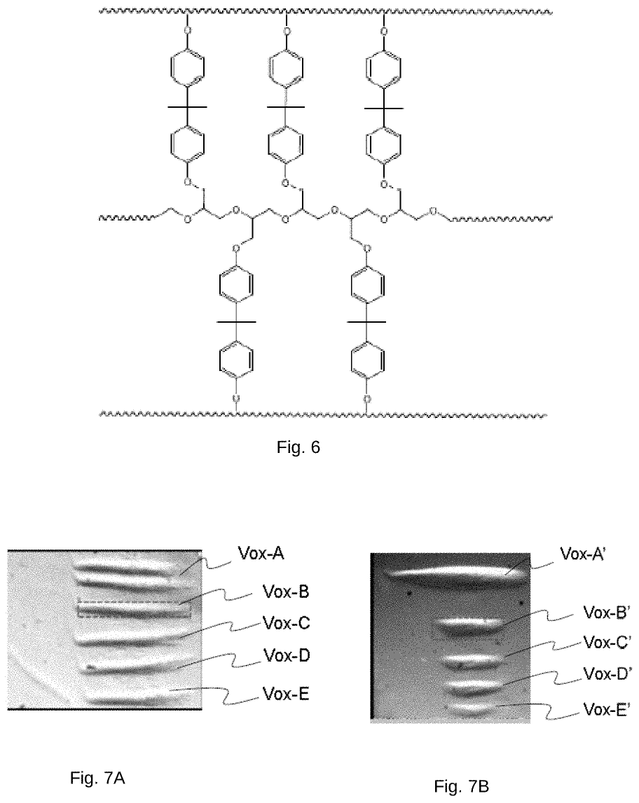

With multifunctional monomers, cross-linked systems, which are insoluble in the initial resin, may be formed as the schematic of FIG. 6 indicates.

Apart from compounds belonging to the family of the epoxies, it is possible to use a high number of monomers described in a synthetic manner a review of which is given in the following reference: Oskar Nuyken & Stephen D. Pask, “Ring-Opening Polymerization—An Introductory Review,” Polymers, 2013, 5, pp. 361-403, doi:10.3390/polym5020361.

As indicated above, a focusing device 13 and a diffuser 14 allowing the depth of field of the laser beam 7 to be controlled and/or modified are placed on the optical path of the laser beam 7.

FIG. 7A shows a plurality of photo-polymerized voxels vox-A, vox-B, vox-C, vox-D and vox-E without diffuser 14 and with various powers of laser beam 7.

FIG. 7B shows a plurality of photo-polymerized voxels vox-A′, vox-B′, vox-C′, vox-D′ and vox-E′ with a diffuser 14 on the optical path of the laser beam 7 and with different powers of laser beam 7.

The use of a suitable 1° and 20° diffuser 14 (a 1° diffuser means that the laser beam 7 has an aperture of 1° on exiting the diffuser 14) allows the size of the photo-polymerized voxels to be varied. However, obviously, the power of the light source 5 must be set so that the power density is identical or as close as possible to that defined for the voxels of smallest size (substantially varying between the square and the cube of the size of the voxel).

Because the composition 11 has a high viscosity (for example, higher than 1.00 Pa·s for 40 vol % of nanoparticles with respect to the volume of the composition), the method according to the present disclosure allows complex three-dimensional objects 3 of at least centimeter size to be designed and produced without needing to make recourse to holding and supporting appendices.

The method according to the present disclosure also allows the time required to produce the three-dimensional object 3 by multi-photon, and notably two-photon, photo-polymerization to be decreased.

Specifically, it is possible to distinguish between an external surface of the three-dimensional object 3 and an internal volume. The optimization then consists in polymerizing locations located in the internal volume (bulk) with a low resolution, defined depending on the object to be printed, and in polymerizing the regions forming the external surface of the three-dimensional object 3 with a high resolution, in order to give the external surface(s) of the three-dimensional object 3 a high-quality surface finish.

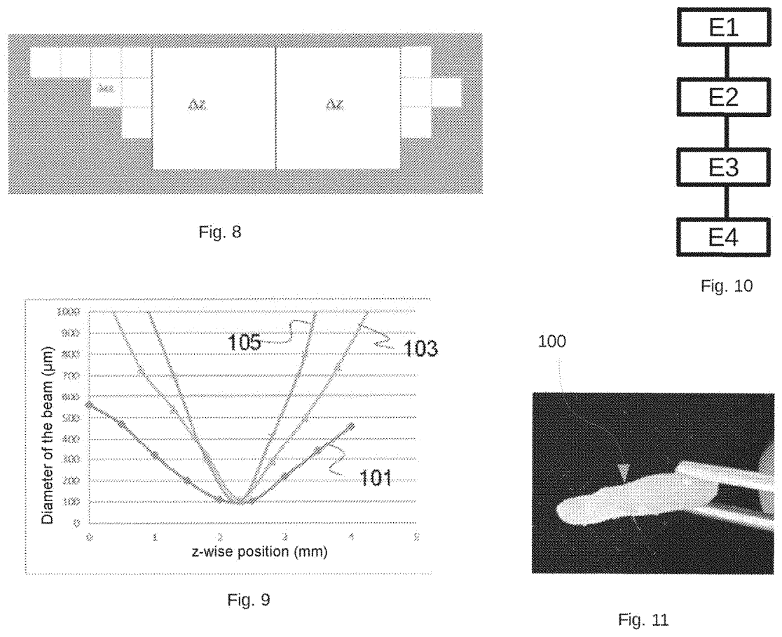

This is schematically shown in FIG. 8. For the sake of simplicity of the description, it is assumed that the voxels are cubes and that, for example, there is at least a first resolution allowing voxels of size Δz to be produced and a second, finer, resolution allowing voxels of smaller size Δzz to be produced, with 10*Δzz=Δz for example.

It will easily be understood that if the voxels inside the three-dimensional object are produced with the resolution Δz and the voxels forming the external surface of the three-dimensional object 3 are produced with the resolution Δzz, the manufacturing time of the three-dimensional object 3 may be significantly decreased.

According to one particular embodiment, the three-dimensional object 3 may be manufactured successively. The internal volume is polymerized from a first composition 11 comprising first fillers 20 taking the form of nanoparticles allowing a first voxel size, with is large with respect to the three-dimensional object 3 to be manufactured, to be obtained. The internal portion of the object is then removed from the first tank 9 comprising the first composition 11. This internal portion is then submerged in a second tank 9 containing a second composition 11 comprising finer nanoparticles than the first composition 11, or even containing no filler 20, for the polymerization of the external surface of the three-dimensional object 3. These first and second successive compositions 11 allow the size of the first and second voxels to be decreased depending on the finish of the three-dimensional object 3 to be formed. The method thus allows localized locations in the internal volume to be polymerized with a lower resolution than the locations forming the external surface of the three-dimensional object 3.

This method thus allows three-dimensional objects 3 the shape of which may be more complex that those accessible with conventional stereolithography methods to be produced easily and rapidly. It is thus possible to envision the manufacture of complex objects having dimensions that are centimeters in size, or even about ten centimeters in size, in a reasonable manufacturing time and without making recourse to holding elements, which would otherwise need to be present in the structure of the three-dimensional object 3.

The method therefore has a decisive advantage relative to single-photon stereolithography, since the thickness of the layer cannot, in generally, be easily modified during the polymerization of a resin layer. Although it is possible to modify the size of the light spot, only two spatial (voxel) parameters may be modified, whereas in the method described here, it is possible to adjust the size of the voxels as a function of three parameters: the diameter of the voxel, and the depth and the power of the light source, to produce an object according to settings taking into account the surface finish of that portion of the three-dimensional object 3 being produced.

According to one particular embodiment, the method may be a bioprinting method. In this case, the composition 11 comprises monomers 12, which are advantageously biocompatible, at least one filler 20 comprising nanoparticles and at least one biological consumable corresponding to a living additional constituent of the filler 20.

By way of non-limiting example, the monomers 12 may be chosen from the following compounds: acrylic monomers, L-lactic acid, glycolic acid, and caprolactones, these compounds possibly being used alone or in combination. The filler 20 comprises nanoparticles that allow the viscosity of the composition 11 to be modified, and furthermore at least one biological consumable corresponding to the additional constituent of the filler 20, such as living cells, for example. According to this particular embodiment, the fillers 20 are therefore composed of nanoparticles that are associated with the living cells, these nanoparticles possibly being in a mixture of collagen and living cells, for example.

A hydrogel is necessary in order to preserve the viability of the cells during printing. By way of non-limiting example, the hydrogel may be chosen from collagen, fibrin, the alginate, chitin, chitosan, hyaluronic acid, poly-(2-hydroxyethyl methacrylate) (PHEMA), polyvinyl alcohol (PVA) and polyethylene glycol (PEG), whether alone or mixed.

With reference to FIG. 10, a method for producing a three-dimensional object 3 is schematically shown.

This method implements an operation E1 of introducing the composition 11 in the polymerization tank 9, the composition 11 comprising at least one monomer 12, at least one filler 20 taking the form of nanoparticles and at least one photoinitiator. When the composition 11 has a high viscosity, this composition 11 may be heated slightly so as to allow its viscosity to be decreased in order to facilitate this introducing operation E1. Moreover, in such a situation, the appearance of air bubbles in the polymerization tank 9 may be prevented by slightly heating the composition 11.

The method then implements an operation E2 of polymerizing, by multi-photon polymerization using the light source 5, in preset locations. During this polymerizing operation E2, the polymerization tank 9 is moved along the axes x, y, z (shown in FIG. 1) so as to allow the focal region of the laser beam 7 allowing the composition 11 to be polymerized to be moved.

Optionally, the method then implements a step E3 of removing the printed three-dimensional object 3 from the polymerization tank 9. In the presence of fillers 20 taking the form of nanoparticles, this removing operation E3 may be carried out in a conventional way using a pair of pincers or indeed a sieve, for example.

Next, and also optionally, the method may implement an operation E4 of removing excess nanoparticles. These excess nanoparticles form, with the unpolymerized monomer 12, a film on the obtained three-dimensional object 3. This may be removed by wiping, using a dip in a bath or even by rinsing with a solvent that solubilizes the unpolymerized monomer 12, this allowing nanoparticles present on the surface to be removed. This removing operation E4 may be carried out at the end of bulk resin printing. In certain cases, the fluidification of the unpolymerized composition 11, and, in particular, of at least one monomer 12, may be achieved by adding liquid monomer 12, this allowing unconverted material to be recycled, or using a conventional solvent of the monomer 12.

In particular, even if the nanoparticles of the filler 20 make mutual contact (maximum filler density) or even form a compact stack, the monomer 12 in liquid form enters into the free spaces and, by polymerizing, binds the nanoparticles around the point where the laser beam 7 is focused. Peripheral nanoparticles, which are not or insufficiently bound by polymerization, are then removed during the removing operation E4. According to the particular embodiment of FIG. 10, this removing operation E4 is carried out by rinsing with a solvent, notably one chosen from ketonic or alcoholic compounds, and notably acetone or even isopropanol.

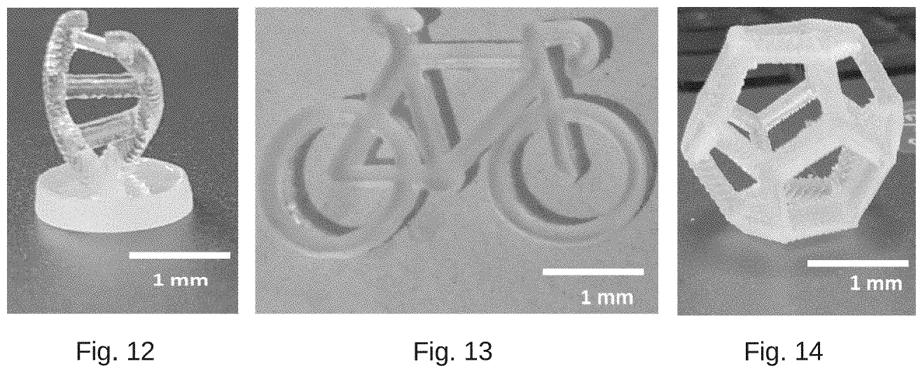

With reference to FIG. 11, a three-dimensional object 3 obtained using this method has been shown. In this figure, the three-dimensional object 3 is of substantially elliptical shape. However, according to other embodiments, and notably when other types of movement of the polymerization tank 9 are used, other shapes, including shapes of higher complexity, may be obtained. Thus, certain objects having geometric shapes of greater or lesser complexity may be obtained using this method. By way of example, FIG. 12 shows a photograph of an object having a complex dodecahedron shape, FIG. 13 shows a photograph of an object having a bicycle shape and FIG. 14 shows a photograph of an object having a double-helix DNA shape. The width scale shown in these figures corresponds to 1 mm (scale of 2.45 cm=1 real mm).

According to one particular embodiment (not shown here) the polymerization tank 9 may comprise a holder, such as, for example, a scaffold, on which the three-dimensional object 3 is manufactured by multi-photon photo-polymerization. The use of such a holder allows the stability of the three-dimensional object 3 to be guaranteed during its manufacture. Specifically, when the manufacturing times of such three-dimensional objects 3 are long, for example longer than 15 seconds, the latter may be lead to move toward the bottom of the polymerization tank 9 according to Stokes law. Such a movement of the object during its manufacture could adversely affect the precision with which this three-dimensional object 3 is manufactured. This movement may therefore be prevented using the holder present in the polymerization tank 9 on which the three-dimensional object 3 is produced.

Specific examples of compositions 11 and of a method for preparing nanoparticles for use as filler 20 are given below.

### Preparation of the Nanoparticles:

The nanoparticles are prepared using a two-step method that consists in obtaining the nanoparticles in powder form then in dispersing them in the monomer 12. This method is notably described in the following documents:
Kulkarni et al “Application of nano-fluids in heating buildings and reducing pollution,” Applied Energy, 2009, 86, pp. 2566-2573;
Longo and Zilio, “Experimental measurement of thermo-physical properties of oxide-water nano-fluids down to ice-point,” Experimental Thermal and Fluid Science, 2011, 35, pp. 1313-1324;
Ho et al, “An experimental investigation of forced convective cooling performance of a microchannel heat sink with Al₂O₃/water nanofluid,” Applied Thermal Engineering, 2010, 30, pp. 96-103; and
Zhang et al, “Effective thermal conductivity and thermal diffusivity of nanofluids containing spherical and cylindrical nanoparticles,” Experimental Thermal and Fluid Science, 2007, 31, pp. 593-599.

In order to ensure the good distribution of the nanoparticles in the composition 11, this composition is stirred for about 1 hour via mechanical stirring and/or via stirring in ultrasound of about 25 kHz optionally for longer periods of time. These stirring times may be increased when the filler content is increased, this possibly corresponding, in particular, locally to “gelling” of the medium.

As indicated above, the use of nanoparticles allows a tailored increase in the viscosity of the composition 11 without needing to take into account the effects of scatting of the light of the laser beam 7.

### Selection of the Monomers 12:

The following monomers are particularly suitable for the method described above:

| | | | Measured | |
|---|---|---|---|---|
| | | | refractive | |
| | | | index (at | |
| Reference | Supplier | Composition | 515 nm) | Viscosity |
| PEGDA 575 | Servilab | Poly(ethyleneglycol) | 1.468 | 0.05 Pa · s |
| | (Sigma) | diacrylate | | |
| TEGDA | Servilab | Tri(ethyleneglycol) | 1.4585 | 0.02 Pa · s |
| | (Sigma) | diacrylate 95% | | |
| PETA | Servilab | Pentaerythritol | 1.484 | 0.60 Pa · s |
| | (Sigma) | tetraacrylate | | |
| HDDA | Servilab | 1,6-hexanediol | 1.456 | 0.02 Pa · s |
| | (Sigma) | diacrylate | | |
| Norland 65 | Thorlab | | 1.499 | 1.20 Pa · s |
| | (Norland) | | | |
| Norland 81 | Thorlab | | 1.523 | 0.30 Pa · s |
| | (Norland) | | | |

The refractive indices were measured using an Abbe refractometer (Kern Optics ORT 1RS Refractometer) calibrated using a calibration oil.

Moreover, the monomers 12 Norland 65 and 81 incorporate a photoinitiator and were used without adding an additional photoinitiator.

### Example of a Composition 11:

| Nature | Type | Supplier |
|---|---|---|
| Monomer | 2-methylmethacrylate | Sigma Aldrich |
| Initiator | Irgacure 651 | Ciba |
| Charge | Silica nanoparticles | Sigma Aldrich |
| | (0.007 μm fused silica | |
| | powder, about 40% of | |
| | the total weight of | |
| | the composition) | |

This composition 11 was polymerized by two-photon polymerization using a Yb:KGW laser 5 that was frequency doubled to 515 nm and pulse durations of 500 fs to obtain an object of substantially cylindrical shape.

Furthermore, the composition 11 has a viscosity that was satisfactory with respect to preventing the movements of the object to be printed during its production and exhibited a small variation in the refractive index of its various components. The composition 11 was also transparent at the preset photo-polymerization wavelength.

### Another Example, Obtainment of Variable Voxels:

An experiment was carried out in order to determine the effects of diffusers 14 placed at the entrance of the objective and allowing a broad range of spatial frequencies to be achieved. Specifically, if the initial laser beam 7 is a plane wave propagating in a certain direction, the diffuser separates this wave into multiple waves that propagate randomly within a characteristic angle of the diffuser 14 (which is related to roughness or “spatial frequency”).

The producing device 1 comprises a light source 5 such as an He/Ne laser of 543 nm wavelength, a long-working-distance objective and a set of various diffusers 14 mounted on a filter wheel.

The measured caustic of the laser beam 7 is plotted in FIG. 9. These measurements allow the influence of the diffusers 14 on the diameter of the laser beam 7 to be determined.

Three curves 101, 103 and 105 are shown in FIG. 9. The curve 101 shows the diameter of the laser beam 7 in μm as a function of the z-wise position in mm without diffuser; the curve 103 shows the same with a 1° diffuser 14, and the curve 105 the same with a 10° diffuser 14.

It may be seen that this method makes it possible to control the depth of field of the Gaussian beam without decreasing the diameter of the laser beam 7 at the focal point and thus to control the dimensions of the voxel.

In the present case, the diameter of the laser beam 7 may reach 100 μm in diameter and a depth of field, defined by an increase in diameter of 2^{0.5}, of about 300 i.e., a ratio between diameter and depth of field of the order of 0.3 (FIG. 3).

1. A method for producing a three-dimensional object, comprising the following operations:
introducing a composition into a polymerization tank; and
polymerizing by multi-photon polymerization using a light source, in predefined locations, the composition, in order to produce the three-dimensional object, the composition comprising at least one monomer, at least one filler and at least one photoinitiator;
wherein the filler comprises nanoparticles.

2. The method for producing a three-dimensional object as claimed in claim 1, wherein the composition has a transmittance per unit length higher than 75% at the emission wavelengths of the light source.

3. The method for producing a three-dimensional object as claimed in claim 1, wherein the composition has a viscosity higher than or equal to 0.30 Pa·s.

4. The method for producing a three-dimensional object as claimed in claim 1, wherein the nanoparticles have an average diameter smaller than or equal to 100 nm.

5. The method for producing a three-dimensional object as claimed in claim 1, wherein the difference in the refractive indices of the nanoparticles and of the monomer is smaller than 0.4.

6. The method for producing a three-dimensional object as claimed in claim 1, wherein the composition comprises from 10 vol % to 70 vol % of nanoparticles with respect to the volume of the composition.

7. The method for producing a three-dimensional object as claimed in claim 1, wherein the fillers comprise a component that is soluble in the monomer.

8. The method for producing a three-dimensional object as claimed in claim 1, wherein the nanoparticles are made of a material chosen from: silica, glass, notably borosilicate glass or soda-lime glass, and an organic material that is insoluble in a constituent resin of the three-dimensional object.

9. The method for producing a three-dimensional object as claimed in claim 1, wherein the monomer is chosen from the following compounds: acrylic monomers, L-lactic acid, glycolic acid, and caprolactones, these compounds possibly being used alone or in combination.

10. The method for producing a three-dimensional object as claimed in claim 9, wherein the filler may furthermore comprise an additional constituent chosen from: living cells, a hydrogel chosen from collagen, fibrin, the alginates, chitin, chitosan, hyaluronic acid, poly-(2-hydroxyethyl methacrylate), polyvinyl alcohol and polyethylene glycol, whether alone or mixed.

11. The method for producing a three-dimensional object as claimed in claim 1, wherein the monomer is an acrylic monomer.

12. The method for producing a three-dimensional object as claimed in claim 11, wherein the acrylic monomer is chosen from poly(ethylene glycol) diacrylates, tri(ethylene glycol) dimethacrylates, pentaerythritol tetraacrylates, 1,6-hexanediol diacrylate, or a combination of these compounds.

13. The method for producing a three-dimensional object as claimed in claim 11, wherein the one or more photoinitiators are chosen from: aromatic ketones, aromatic derivatives, eosin Y, or other xanthene dyes.

14. The method for producing a three-dimensional object as claimed in claim 1, wherein the composition comprises at least one epoxy monomer.

15. The method for producing a three-dimensional object as claimed in claim 14, wherein the photoinitiator is an onium salt.

16. The method for producing a three-dimensional object as claimed in claim 1, in which method the multi-photon polymerization is carried out using a laser beam, wherein the polymerization spatial resolution is set by placing an optical diffuser, notably of between 1° and 20°, in the laser beam.

17. The method for producing a three-dimensional object as claimed in claim 1, in which method the three-dimensional object comprises an external surface and an internal volume, wherein localized locations in the internal volume are polymerized with a lower resolution than locations forming the external surface of the three-dimensional object.

18. The method for producing a three-dimensional object as claimed in claim 17, wherein various segments of the three-dimensional object are successively polymerized in various tanks each containing a specific composition allow a predefined voxel size, or even predefined functionalities, to be obtained.

19. The method for producing a three-dimensional object as claimed in claim 17, wherein the internal volume is polymerized in a first tank containing a first composition comprising first fillers taking the form of nanoparticles allowing a first voxel size to be obtained and the external portion of the three-dimensional object is polymerized in a second tank containing a second composition comprising second fillers taking the form of nanoparticles or no fillers allowing a second voxel size smaller than the first voxel size to be obtained.

20. A device for producing a three-dimensional object by multi-photon and notably two-photon photo-polymerization, wherein the device comprises:
a light source that emits a laser beam,
a polymerization tank containing a composition comprising:
at least one monomer,
at least one filler comprising nanoparticles, the composition having a viscosity higher than or equal to 0.30 Pa·s, and
at least one photoinitiator,
a device for focusing the laser beam and for setting its numerical aperture,
a moving unit in order to allow the focal region of the laser beam to be moved inside the tank to preset locations in order to produce the three-dimensional object, and
a polymerization-resolution setter comprising at least one optical diffuser moveably mounted on a holder so as to be placeable on the optical path or outside of the laser beam, in order to set the polymerization resolution.

21. The device of claim 20, wherein the composition has a transmittance per unit length preferably higher than 75% at the emission wavelengths of the light source.
